(19) United States
(12) Patent Application Publication
(10) Pub. No.: US 2017/0144312 A1
(43) Pub. Date: May 25, 2017

(54) ROBOTIC SURGICAL FINGER AND CONTROLLER WITH TACTILE FEEDBACK AND ROBOTIC HAND USING THE SAME

(71) Applicant: UMM AL-QURA UNIVERSITY, Makkah (SA)

(72) Inventors: JAMAL TALAAT HAMDI, MAKKAH (SA); SHADI MOHAMMAD MUNSHI, MAKKAH (SA); SUFYAN AZAM, KUDAI MAKKAH (SA)

(21) Appl. No.: 14/952,544

(22) Filed: Nov. 25, 2015

(51) Int. Cl. B25J 13/02; A61B 90/00; A61B 34/00; B25J 13/08; A61B 34/37

(52) U.S. Cl. CPC B25J 13/025; B25J 13/081; A61B 34/37; A61B 34/76; A61B 90/06; A61B 2090/067; A61B 2090/065; A61B 2560/0425

(57) ABSTRACT

The robotic surgical finger and controller with tactile feedback is a remotely controllable robotic finger for surgical examination and procedures, for example, that provides real-time temperature and pressure feedback to the user, as well as friction feedback for detecting texture, slippage and the like. The robotic finger includes a plurality of joined segments simulating a human finger, and a sensor module mounted on each of the segments. A control sheath receives a finger of a user's hand and includes at least one joint angle sensor in communication with a plurality of servomotors for driving angular movement of the plurality of segments of the robotic finger. A plurality of tactile feedback modules are further mounted in the control sheath for providing temperature, pressure and friction feedback signals to the user's finger based on measurements made by the sensor modules of the robotic finger. A hand using multiples fingers is further provided.

### BACKGROUND OF THE INVENTION

1. Field of the Invention

The present invention relates to medical instruments and appliances, and particularly to a robotic finger and controller with tactile feedback that can be used to provide the surgeon with some measure of tactile feedback when performing laparoscopic, robotic, and other minimally invasive procedures.

2. Description of the Related Art

Traditionally, surgeons have performed dissections using their index fingers to determine and maneuver into the proper plane for the procedure. Typical laparoscopic, robotic and remote surgical systems are very useful, but unfortunately, do not provide the surgeon with the traditional and very effective use of his or her finger. Laparoscopic and robotic surgical systems are growing in use for certain procedures, but they remain limited due to the lack of haptic and tactile feedback to the surgeon.

Surgeons are typically trained to feel tissue characteristics, to identify pathologic conditions by touch, and, as noted above, to use touch in order to properly maneuver and position instruments during dissections. Although haptic feedback is known for various types of robotic systems, typical haptic feedback systems are neither sensitive enough, nor do they provide enough sensory information for effective use in surgical examination and procedures.

Thus, a robotic surgical finger and controller with tactile feedback and robotic hand using the same solving the aforementioned problems is desired.

### SUMMARY OF THE INVENTION

The robotic surgical finger and controller with tactile feedback is a remotely controllable robotic finger for surgical examination and procedures that provides real-time temperature and pressure feedback to the user, as well as providing tactile or friction feedback for detecting texture, slippage and the like. The robotic finger includes a plurality of joined segments simulating a human finger. Each segment is pivotally joined to at least one adjacent one of the plurality of segments such that the segments are bendable with respect to one another in a manner similar to the natural bending of the segments of a human finger. A sensor module is mounted on each of the segments. Each sensor module includes a pressure sensor, a temperature sensor and a friction sensor. Additionally, a plurality of servomotors are in communication with the plurality of segments for selectively driving and controlling angular movement of the segments.

A control sheath includes a sheath housing adapted for receiving the medical practitioner's finger. At least one joint angle sensor, such as a piezoresistive sensor or the like, is mounted in the sheath housing and is positioned adjacent at least one joint of the finger of the user's hand. The at least one joint angle sensor is in communication with the plurality of servomotors such that movement of at least one joint of the finger of the user's hand is detected by the at least one joint angle sensor and the plurality of servomotors drive and control angular movement of the plurality of segments of the robotic finger to simulate the movement of the at least one joint of the finger of the user's hand.

A plurality of tactile feedback modules are also mounted in the sheath housing. Each tactile feedback module is in communication with a corresponding one of the plurality of sensor modules for providing temperature and friction sensations to the finger of the user's hand corresponding to temperature and friction measured by the plurality of sensor modules. Additionally, a plurality of fillable bladders are also mounted in the sheath housing. A pneumatic controller is in fluid communication with the plurality of finable bladders, and the pneumatic controller is in communication with the plurality of sensor modules for selectively filling each of the fillable bladders to provide pressure sensations to the finger of the medical practitioner's hand corresponding to pressure measured by the plurality of sensor modules. Preferably, each of the tactile feedback modules and each of the finable bladders are adapted for at least partially wrapping around a corresponding segment of the finger of the medical practitioner's hand.

Multiple robotic fingers may be used in a robotic grasping tool. Each robotic finger is mounted on a support, and multiple control sheaths are provided for the user to wear on corresponding fingers. For example, a robotic finger having two segments and a robotic finger having three segments may be mounted on a support to simulate the user's thumb and index finger. A corresponding control sheath for the user's thumb and a control sheath for the user's index finger could then be worn for respectively controlling the robotic thumb and robotic index finger. The user may then make a pinching or grasping movement for pinching or grasping with the robotic thumb and robotic index finger of the robotic grasping tool. Similarly, five such control sheaths may be worn, either separately or integrated into a glove, for controlling five corresponding robotic fingers mounted on a central support, forming a robotic hand.

These and other features of the present invention will become readily apparent upon further review of the following specification and drawings.

### BRIEF DESCRIPTION OF THE DRAWINGS

FIG. 1 is a schematic diagram of a robotic surgical finger and controller with tactile feedback according to the present invention.

FIG. 2 is an environmental side view of a control sheath of the robotic surgical finger and controller with tactile feedback according to the present invention, the sheath being broken away to show details thereof.

FIG. 3 is a perspective view of a robotic grasping tool having a plurality of robotic fingers and controller with tactile feedback according to the present invention.

FIG. 4 is a perspective view of another embodiment of a grasping tool having a plurality of robotic fingers and controller with tactile feedback according to the present invention,

FIG. 5 is a side view of an alternative embodiment of the control sheath of the robotic surgical finger and controller with tactile feedback of FIG. 2.

Similar reference characters denote corresponding features consistently throughout the attached drawings.

### DETAILED DESCRIPTION OF THE PREFERRED EMBODIMENTS

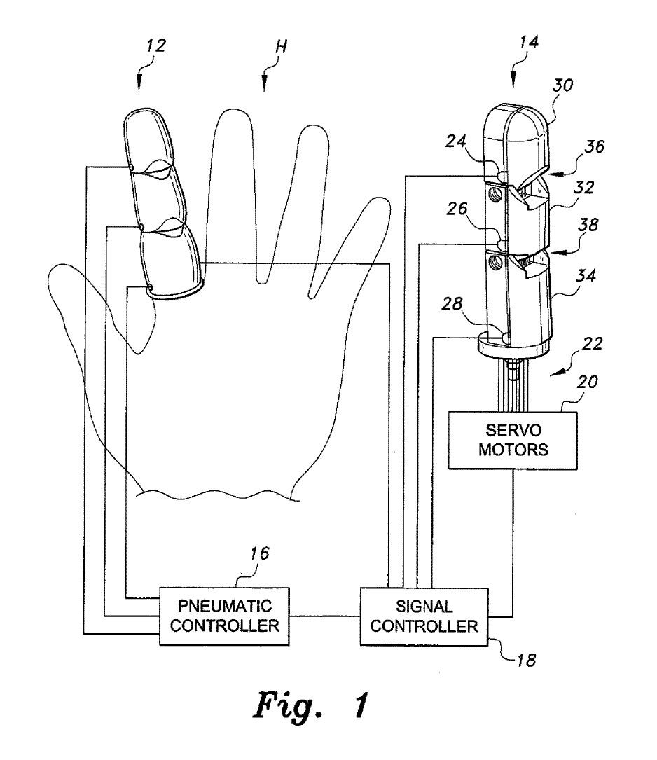

The robotic surgical finger and controller with tactile feedback 10 is a remotely controllable robotic finger for surgical examination and procedures, for example, that provides real-time temperature and pressure feedback to the user, as well as providing frictional feedback for detecting texture, slippage and the like. As shown in FIG. 1, the robotic finger 14 includes a plurality of joined segments 30, 32, 34 simulating a human finger. Each of the segments 30, 32, 34 is articulated or pivotally joined to at least one adjacent segment 30, 32, 34 such that the segments bend with respect to one another in a manner similar to the natural bending or articulation of the segments of a human finger. It should be understood that joints 36, 38 are shown in FIG. 1 for exemplary purposes only, and that segments 30, 32, 34 may be joined to one another by any suitable type of joint, hinge, pivot or the like. Further, it should be understood that although the robotic finger 14 of FIG. 1 includes three segments 30, 32, 34 pivotally joined to one another by joints 36, 38, resembling a human index finger, this particular configuration is shown for exemplary purposes only and the robotic finger 14 may include any desired number of segments and may have any desired overall relative dimensions and configuration. For purposes of laparoscopic or robotic surgery, the robotic finger 14 is preferably dimensioned and configured to permit introduction through a conventional 10 mm or 12 mm cannula.

A sensor module 24, 26, 28 is respectively mounted on each of the segments 30, 32, 34. Each sensor module 24, 26, 28 includes a pressure sensor, a temperature sensor and a friction sensor. It should be understood that any suitable type of pressure, temperature and friction sensors may be used. Such sensors used in robotic systems for haptic feedback are well known in the art. For example, U.S. Pat. No. 8,390,438, which is hereby incorporated by reference in its entirety, teaches pressure/force, temperature, and friction/texture sensors for purposes of haptic feedback in a robotic medical system. It should be understood that any additional type of sensor may also be used in the sensor modules, such as accelerometers or the like.

A plurality of servomotors 20 are in communication with the plurality of segments 30, 32, 34, via interconnecting wires 22 or the like, for selectively driving and controlling angular movement of the segments 30, 32, 34 in a conventional manner. Such servomotor control in robotic systems is well known in the art, and it should be understood that selective driving and control of the angular movement of the segments 30, 32, 34 may be effected by any suitable robotic drive system. Such systems are, for example, shown in U.S. Pat. Nos. 6,296,635 and 5,447,403, each of which is hereby incorporated by reference in its entirety.

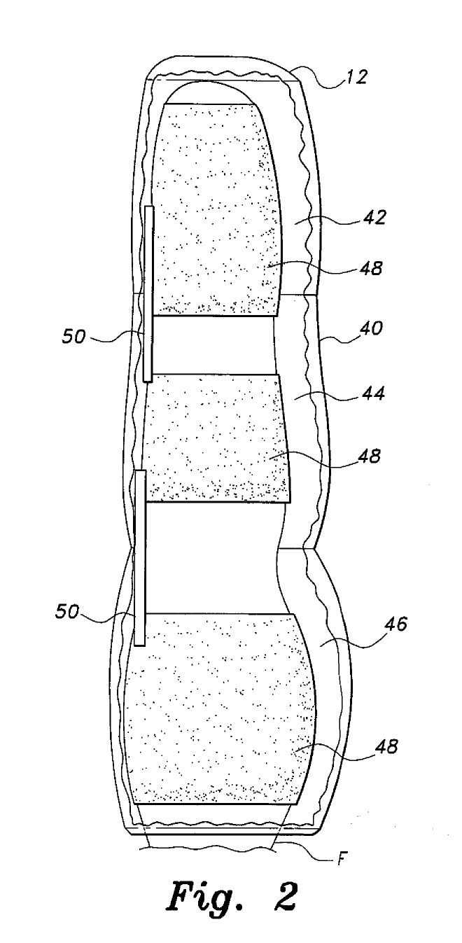

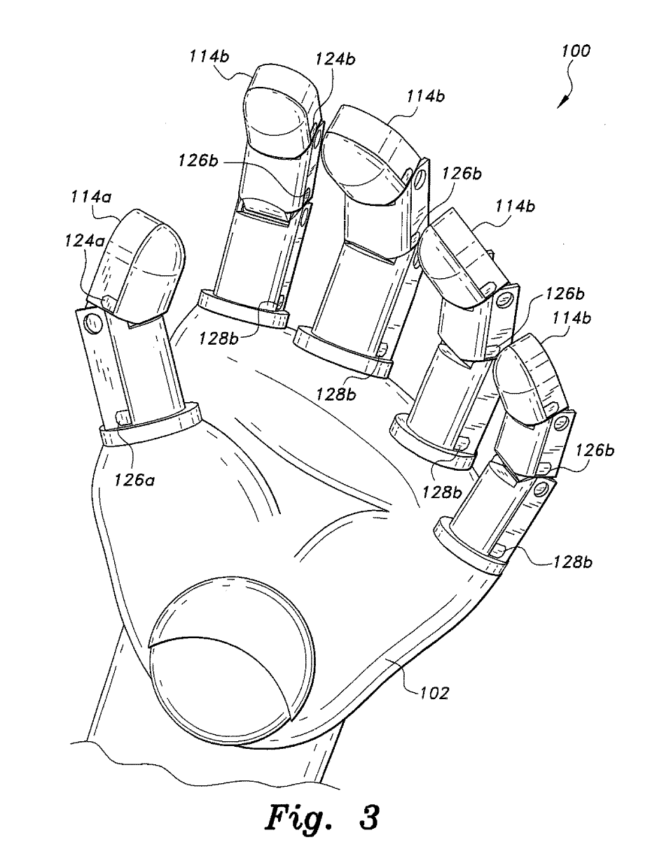

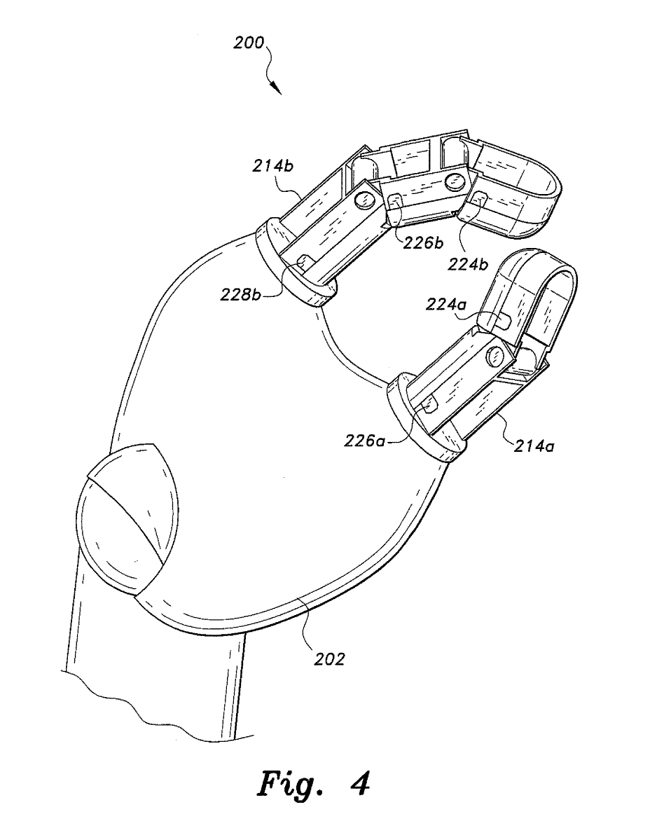

In order to control the robotic finger 14, the user wears a control sheath 12 on a finger F of his or her hand H. As best shown in FIG. 2, the control sheath 12 includes a sheath housing 40 dimensioned and configured for receiving the user's finger F. At least one joint angle sensor 50, such as a piezoresistive sensor or the like, is received in the sheath housing 40 and is positioned adjacent at least one joint of the finger F. In the example of the robotic finger 14 shown in FIG. 1, as described above, three segments 30, 32, 34 are provided for simulating a user's index finger. The control sheath 12 and the robotic finger 14 are both configured for the same type of finger; i.e., for an index finger, for example, the sheath housing 40 is configured to match a human index finger (having three finger segments and two joint angle sensors 50 properly positioned within the sheath housing 40 to rest against the two joints of the user's index finger). As will be described below with particular reference to the embodiments of FIGS. 3 and 4, the system may be configured for use with other types of fingers, including a human thumb, which only has two segments and one joint therebetween.

The at least one joint angle sensor 50 is in communication with the plurality of servomotors 20 via the signal controller 18, such that movement of at least one joint of the user's finger F is detected by the at least one joint angle sensor 50. The signal controller 18 measures the degree of angular movement of the joint(s) of the user's finger F and transmits control signals to the servomotors 20 for reproducing an identical movement in the segments 30, 32, 34 of robotic finger 14. It should be understood that the signal controller 18 may be any suitable type of microcontroller, processor, programmable logic controller or the like.

A plurality of tactile feedback modules 48 are also mounted in the sheath housing 40. Each tactile feedback module 48 is preferably dimensioned and configured to at least partially wrap around a corresponding segment of the user's finger F. Each tactile feedback module 48 is in communication with a corresponding one of the plurality of sensor modules 24, 26, 28, via interconnection through the signal controller 18 for providing temperature and friction/texture sensations to the user's finger F corresponding to temperature and friction measured by the plurality of sensor modules 24, 26, 28. It should be understood that any suitable type of haptic/tactile feedback modules for delivering temperature and friction/texture sensations to the user may be utilized.

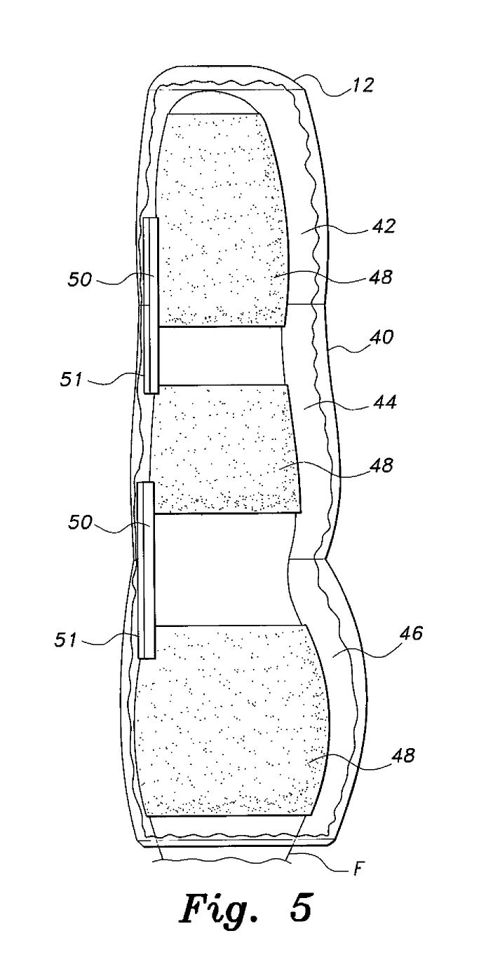

Additionally, a plurality of fillable bladders 42 are also mounted in the sheath housing 40. Each of the fillable bladders 42 also is dimensioned and configured to at least partially wrap around a corresponding segment of the user's finger F. A pneumatic controller 16 is in fluid communication with the plurality of fillable bladders 42, and the pneumatic controller 16 is also in communication with the plurality of sensor modules 24, 26, 28, via interconnection with the signal controller 18 for selectively inflating and deflating each of the fillable bladders 42 to provide pressure/force sensations to the user's finger F corresponding to pressure measured by the plurality of sensor modules 24, 26, 28. It should be understood that any suitable type of pneumatic controller may be used in combination with the signal controller 18 for the selective inflating and deflating of finable bladders 42, depending on pressure measured by the sensor modules 24, 26, 28. In the alternative embodiment of FIG. 5, constrainers 51 have been added to constrain the surgeon's finger movement to prevent accidental excess pressure. Constrainers 51 may be pneumatic or any other fluid-based constraint means to prevent the application of extra or excess pressure from the robotic finger on vital structures.

In the embodiment of FIG. 3, multiple robotic fingers are used in a robotic grasping tool 100. In FIG. 3, the robotic grasping tool 100 is configured as a full robotic hand, including four three-segment fingers 114b, similar to the robotic finger 14, each with corresponding sensor modules 124b, 126b, 128b, and one two-segment finger 114a, simulating a two-segment human thumb, with corresponding sensor modules 124a, 126a. Each robotic finger 114a, 114b is mounted on a support 102, configured similarly to a human palm. In order to control the robotic grasping tool 100, multiple control sheaths are provided for the user to wear on corresponding fingers of his or her hand. In the example of FIG. 3, five such sheaths, as described above with respect to FIG. 2, may be integrated into a glove to be worn on the user's hand.

In the alternative configuration of FIG. 4, only two such robotic fingers 214a, 214b are used to form robotic grasping tool 200. In this example, robotic finger 214a is, once again, a two-segment robotic finger (with corresponding sensor modules 224a, 226a) simulating a human thumb, and robotic finger 214b is a three-segment robotic finger (with corresponding sensor modules 224b, 226b, 228b), similar to robotic finger 14, simulating an index finger. Robotic fingers 214a, 214b are each mounted on a support 202. As described above, a corresponding control sheath for the user's thumb and a control sheath for the user's index finger may then be worn for respectively controlling the robotic thumb 214a and robotic index finger 214b. The user may then make a pinching or grasping movement for pinching or grasping with the robotic thumb 214a and robotic index finger 214b of the robotic grasping tool 200.

It is to be understood that the present invention is not limited to the embodiments described above, but encompasses any and all embodiments within the scope of the following claims.

We claim:

1. A robotic finger with tactile feedback, comprising:
a robotic finger having:
a plurality of segments, each of the segments being pivotally joined to at least one adjacent segment;
a plurality of sensor modules, each of the sensor modules being mounted on a corresponding one of segments, each of the sensor modules including a pressure sensor, a temperature sensor, and a friction sensor; and
a plurality of servomotors in communication with the plurality of segments for selectively driving and controlling angular movement thereof; and
a control sheath having:
a sheath housing adapted for receiving a finger of a user's hand;
at least one joint angle sensor mounted in the sheath housing and positioned adjacent at least one joint of the finger of the user's hand, the at least one joint angle sensor being in communication with the plurality of servomotors such that movement of at least one joint of the finger of the user's hand is detected by the at least one joint angle sensor, and in response, the plurality of servomotors drive and control angular movement of the plurality of segments of said robotic finger to simulate the movement of the at least one joint of the finger of the user's hand;
a plurality of tactile feedback modules mounted in the sheath housing, wherein each of the tactile feedback modules is in communication with a corresponding one of the plurality of sensor modules for providing temperature and friction sensations to the finger of the user's hand corresponding to temperature and friction measured by the plurality of sensor modules;
a plurality of fillable bladders mounted in the sheath housing; and
a pneumatic controller in fluid communication with the plurality of fillable bladders, the pneumatic controller being in communication with the plurality of sensor modules for selectively filling each of the fillable bladders to provide pressure sensations to the finger of the user's hand corresponding to pressure measured by the plurality of sensor modules.

2. The robotic finger as recited in claim 1, wherein the at least one joint angle sensor comprises a piezoresistive sensor.

3. The robotic finger as recited in claim 1, wherein each said tactile feedback module is adapted for at least partially wrapping around a corresponding segment of the finger of the user's hand.

4. The robotic finger as recited in claim 3, wherein each said fillable bladder is adapted for at least partially wrapping around a corresponding one of the segments of the finger of the user's hand.

5. A robotic grasping tool and controller with tactile feedback, comprising:
a support;
a plurality of robotic fingers mounted on the support, each of the robotic fingers having:
a plurality of segments, each of the segments being pivotally joined to at least one adjacent segment;
a plurality of sensor modules, each of the sensor modules being mounted on a corresponding one of the segments, each of the sensor modules including a pressure sensor, a temperature sensor, and a friction sensor; and.
a plurality of servomotors in communication with the plurality of segments for selectively driving and controlling angular movement thereof; and
a plurality of control sheaths, each of the control sheath having:
a sheath housing adapted for receiving a corresponding finger of a user's hand;
at least one joint angle sensor mounted in the sheath housing and positioned adjacent at least one joint of the corresponding finger of the user's hand, the at least one joint angle sensor being in communication with the plurality of servomotors such that movement of at least one joint of the finger of the user's hand is detected by the at least one joint angle sensor, and in response, the plurality of servomotors drive and control angular movement of the plurality of segments of the corresponding robotic finger to simulate the movement of the at least one joint of the corresponding finger of the user's hand;
a plurality of tactile feedback modules mounted in the sheath housing, each of the tactile feedback modules being in communication with a corresponding one of the plurality of sensor modules for providing temperature and friction sensations to the finger of the user's hand corresponding to temperature and friction measured by the plurality of sensor modules;
a plurality of fillable bladders mounted in the sheath housing; and
a pneumatic controller in fluid communication with the plurality of fillable bladders, the pneumatic controller being in communication with the plurality of sensor modules for selectively filling each of the fillable bladders to provide pressure sensations to the corresponding finger of the user's hand reflecting pressure measured by the plurality of sensor modules.

6. The robotic grasping tool as recited in claim 5, wherein the at least one joint angle sensor comprises a piezoresistive sensor.

7. The robotic grasping tool as recited in claim 5, wherein each said tactile feedback module is adapted for at least partially wrapping around a corresponding segment of the finger of the user's hand.

8. The robotic grasping tool as recited in claim 7, wherein each said fillable bladder is adapted for at least partially wrapping around a corresponding one of the segments of the finger of the user's hand.
